(19) United States
(12) Patent Application Publication
(10) Pub. No.: US 2022/0416316 A1
(43) Pub. Date: Dec. 29, 2022

(54) Flame Retardants For Battery Electrolytes

(71) Applicant: Albemarle Corporation, Charlotte, NC (US)

(72) Inventors: Zhongxin Ge, Baton Rouge, LA (US); Tse-Chong Wu, Baton Rouge, LA (US); Sascha Joerg Welz, Phoenix, AZ (US); Mark Timothy Bennett, Baton Rouge, LA (US); Yunqi Liu, Baton Rouge, LA (US)

(21) Appl. No.: 17/777,357

(22) PCT Filed: Nov. 18, 2020
(86) PCT No.: PCT/US2020/060943

§ 371 Date: May 17, 2022

Related U.S. Application Data
(60) Provisional application No. 62/936,706, filed on Nov. 18, 2019.

(51) Int. Cl. H01M 10/42; H01M 10/0525; H01M 10/0567; H01M 10/0568; H01M 10/0569

(52) U.S. Cl. CPC H01M 10/4235; H01M 10/0525; H01M 10/0567; H01M 10/0568; H01M 10/0569; H01M 2300/0051

(57) ABSTRACT

This invention provides nonaqueous electrolyte solutions for lithium batteries. The nonaqueous electrolyte solutions comprise a liquid electrolyte medium; a lithium-containing salt; and at least one oxygen-containing brominated flame retardant.

### TECHNICAL FIELD

This invention relates to brominated flame retardants for electrolyte solutions for batteries.

### BACKGROUND

One of the components impacting the safety of lithium-ion batteries is their use of flammable solvents in the lithium-containing electrolyte solutions. Inclusion of a flame retardant in the electrolyte solution is one way to mitigate the flammability of these solutions. For a flame retardant to be a suitable component of an electrolyte solution, solubility in the electrolyte is needed, along with electrochemical stability over the range of battery operation, and minimal negative effect on battery performance. Negative effects on battery performance can include reduced conductivity and/or chemical instability to the active material.

What is desired is a flame retardant that can effectively suppress the flammability of lithium ion batteries with minimal impact to the electrochemical performance of the lithium ion battery at a reasonable cost.

### SUMMARY OF THE INVENTION

This invention provides nonaqueous electrolyte solutions for lithium batteries which contain at least one oxygen-containing brominated flame retardant. In the presence of the oxygen-containing brominated flame retardant(s), fires are extinguished in these nonaqueous electrolyte solutions, at least under laboratory conditions.

An embodiment of this invention is a nonaqueous electrolyte solution for a lithium battery, which solution comprises i) a liquid electrolyte medium; ii) a lithium-containing salt; and iii) at least one oxygen-containing brominated flame retardant. The oxygen-containing brominated flame retardant is a) at least one brominated noncyclic ether, b) a brominated cyclic diether, c) a brominated noncyclic carbonate, or d) a brominated cyclic carbonate. In the brominated noncyclic carbonate, at least one hydrocarbyl group has at least one unsaturated carbon-carbon bond.

Another embodiment of this invention is a nonaqueous electrolyte solution for a lithium battery, which solution comprises i) a liquid electrolyte medium; ii) a lithium-containing salt; and iii) at least one oxygen-containing brominated flame retardant. The oxygen-containing brominated flame retardant is selected from the group consisting of 1-bromo-2-methoxyethane, 1-bromo-3-methoxypropane, 2-bromo-1,1-dimethoxyethane, 2-bromo-1,4-dimethoxybenzene, 1-bromo-2-(methoxymethoxy)ethane, 1-bromovinyl ethyl ether, 1,2-dibromo-3-methoxy-1-propene, 1,2-dibromo-3-ethoxy-1-propene, di(ethylene glycol) dibromovinyl ether, 4-bromo-1,3-dioxolane, 2-bromomethyl-1,3-dioxolane, 2-dibromomethyl-1,3-dioxolane, 2-tribromomethyl-1,3-dioxolane, 2,2-bis(bromomethyl)-1,3-dioxolane, 2-(bromomethyl)-1,4-dioxane, 5,5-bis(bromomethyl)-2-methyl-1,3-dioxane, 5,5-bis(bromomethyl)-2-ethyl-1,3-dioxane, 3-bromo-2-propenyl methyl carbonate, 2,3-dibromo-2-propenyl methyl carbonate, 2,3,3-tribromo-2-propenyl methyl carbonate, 3-bromo-2-propenyl ethyl carbonate, 2,4-dibromophenyl methyl carbonate, bis(2,3-dibromo-2-propenyl) carbonate, 4-bromo-1,3-dioxol-2-one, 4,5-dibromo-1,3-dioxol-2-one, 4-bromomethyl-1,3-dioxol-2-one, 4,4-bis(bromomethyl)-1,3-dioxol-2-one, and 4,5-bis(bromomethyl)-1,3-dioxol-2-one.

These and other embodiments and features of this invention will be still further apparent from the ensuing description and appended claims.

### FURTHER DETAILED DESCRIPTION OF THE INVENTION

Throughout this document, the phrase “electrolyte solution” is used interchangeably with the phrase “nonaqueous electrolyte solution”.

The liquid electrolyte medium is comprised of one or more solvents that typically form the liquid electrolyte medium for lithium electrolyte solutions used in lithium batteries, which solvents are polar and aprotic, stable to electrochemical cycling, and preferably have low viscosity. These solvents usually include noncyclic carbonic acid esters, cyclic carbonic acid esters, ethers, sulfur-containing compounds, and esters of boric acid.

The solvents that can form the liquid electrolyte medium in the practice of this invention include ethylene carbonate (1,3-dioxolan-2-one), dimethyl carbonate, ethyl methyl carbonate, diethyl carbonate, dioxolane, dimethoxy ethane (glyme), tetrahydrofuran, methanesulfonyl chloride, ethylene sulfite, 1,3-propylene glycol boric ester, and mixtures of any two or more of the foregoing.

Preferred solvents include ethylene carbonate, ethyl methyl carbonate, and mixtures thereof. More preferred are mixtures of ethylene carbonate and ethyl methyl carbonate, especially at volume ratios of ethylene carbonate:ethyl methyl carbonate ratios of about 20:80 to about 40:60, more preferably about 25:75 to about 35:65.

Suitable lithium-containing salts in the practice of this invention include lithium chloride, lithium bromide, lithium iodide, lithium perchlorate, lithium nitrate, lithium thiocyanate, lithium aluminate, lithium tetrachloroaluminate, lithium tetrafluoroaluminate, lithium tetraphenylborate, lithium tetrafluoroborate, lithium bis(oxolato)borate (LiBOB), lithium di(fluoro)(oxalato)borate, lithium hexafluorophosphate, lithium hexafluoroarsenate, lithium hexafluoroantimonate, lithium titanium oxide, lithium manganese oxide, lithium cobalt oxide (LiCoO₂), lithium nickel oxide (LiNiO₂), lithium alkyl carbonates in which the alkyl group has 1 to 6 carbon atoms, lithium methyl sulfonate, lithium trifluoromethylsulfonate, lithium pentafluoroethylsulfonate, lithium pentafluorophenylsulfonate, lithium fluorosulfonate, lithium bis(trifluoromethylsulfonyl)imide, lithium bis(pentafluoroethylsulfonyl)imide, lithium (ethylsulfonyl)(trifluoromethylsulfonyl)imide, and mixtures of any two or more of the foregoing. Preferred lithium-containing salts include lithium hexafluorophosphate, lithium di(fluoro)(oxolato)borate, and lithium bis(oxolato)borate.

Typical concentrations for the lithium-containing salt in the electrolyte solution are in the range of about 0.1 M to about 2.5 M, preferably about 0.5 M to about 2 M, more preferably about 0.75 M to about 1.75 M, and still more preferably about 0.95 M to about 1.5 M. When more than one lithium-containing salt forms the lithium-containing electrolyte, the concentration refers to the total concentration of all of the lithium-containing salts present in the electrolyte solution.

The electrolyte solution can contain other salts in addition to lithium salts, unless such other salt(s) materially degrade either the performance of the battery for the desired application, or the flame retardancy of the electrolyte solution. Suitable electrolytes other than lithium salts include other alkali metal salts, e.g., sodium salts, potassium salts, rubidium salts, and cesium salts, and alkaline earth metal salts, e.g., magnesium salts, calcium salts, strontium salts, and barium salts. In some aspects, the salts in the non-aqueous electrolyte solution are only one or more lithium salts.

Suitable alkali metal salts that can be present in the electrolyte solution include sodium salts such as sodium chloride, sodium bromide, sodium iodide, sodium perchlorate, sodium nitrate, sodium thiocyanate, sodium aluminate, sodium tetrachloroaluminate, sodium tetrafluoroaluminate, sodium tetraphenylborate, sodium tetrafluoroborate, and sodium hexafluorophosphate; and potassium salts such as potassium chloride, potassium bromide, potassium iodide, potassium perchlorate, potassium nitrate, potassium thiocyanate, potassium aluminate, potassium tetrachloroaluminate, potassium tetrafluoroaluminate, potassium tetraphenylborate, potassium tetrafluoroborate, and potassium hexafluorophosphate.

Suitable alkaline earth metal salts that can be present in the electrolyte solution include magnesium salts such as magnesium chloride, magnesium bromide, magnesium iodide, magnesium perchlorate, magnesium nitrate, magnesium thiocyanate, magnesium aluminate, magnesium tetrachloroaluminate, magnesium tetrafluoroaluminate, magnesium tetraphenylborate, magnesium tetrafluoroborate, and magnesium hexafluorophosphate; and calcium salts such as calcium chloride, calcium bromide, calcium iodide, calcium perchlorate, calcium nitrate, calcium thiocyanate, calcium aluminate, calcium tetrachloroaluminate, calcium tetrafluoroaluminate, calcium tetraphenylborate, calcium tetrafluoroborate, and calcium hexafluorophosphate.

In the practice of this invention, the brominated flame retardant is miscible with the liquid medium of the nonaqueous electrolyte solution, where “miscible” means that the brominated flame retardant does not form a separate phase from the electrolyte solution. More specifically, a brominated flame retardant is miscible if it forms a single phase in a mixture of 30 wt % ethylene carbonate and 70 wt % ethyl methyl carbonate which contains 1.2 M lithium hexafluorophosphate, after 24 hours of shaking in a mechanical shaker, and no separate phase is formed after the shaking is stopped, and the brominated flame retardant does not precipitate from, or form a suspension or slurry in, the nonaqueous electrolyte solution. It is recommended and preferred that the brominated flame retardant does not cause the precipitation of, or formation of a suspension or slurry of, any of the other components of the nonaqueous electrolyte solution.

In the practice of this invention, the oxygen-containing brominated flame retardants generally have a bromine content of about 35 wt % or more based on the weight of the oxygen-containing brominated flame retardant and a boiling point of about 75° C. or higher, preferably about 95° C. or higher. The oxygen-containing brominated flame retardants in the practice of this invention have a bromine content in the molecule that ranges from about 35 wt % to about 80 wt %, more preferably about 40 wt % to about 75 wt %.

The boiling point of the brominated flame retardants in this invention are about 75° C. or more, preferably about 95° C. or more, and typically range from about 75° C. to about 450° C., preferably from about 95° C. to about 425° C., more preferably from about 100° C. to about 410° C. The boiling points described throughout this document are at standard temperature and pressure (standard conditions) unless otherwise stated.

The brominated flame retardants used in the practice of this invention are generally polar and aprotic, stable to electrochemical cycling, and preferably have low viscosities.

In the practice of this invention, a flame retardant amount in the nonaqueous electrolyte solution means enough flame retardant is present that the solution passes the modified horizontal UL-94 test described below. The flame retardant amount is often different for different brominated flame retardants, but is usually about 20 wt % flame retardant molecules, preferably about 25 wt % or more flame retardant molecules, relative to the total weight of the nonaqueous electrolyte solution. Similarly, the flame retardant amount in terms of bromine content is usually about 10 wt % or more bromine (atoms), preferably about 11 wt % or more, relative to the total weight of the nonaqueous electrolyte solution.

The oxygen-containing brominated flame retardants of this invention share some overall characteristics. In these brominated flame retardants, the bromine content is about 35 wt % or more, preferably about 35 wt % to about 80 wt %, more preferably about 40 wt % to about 75 wt %, relative to the total weight of the flame retardant molecule; there are typically one to about five bromine atoms in the oxygen-containing brominated flame retardant molecule; and there are about three to about ten carbon atoms in the oxygen-containing brominated flame retardant molecule.

In some embodiments, the oxygen-containing brominated flame retardant is a brominated noncyclic ether. The brominated noncyclic ether generally has one or more oxygen atoms, typically one to about three oxygen atoms, and preferably has two oxygen atoms. The hydrocarbyl groups of the brominated noncyclic ether are alkyl, alkenyl, aryl, or ar-alkyl groups. When the hydrocarbyl groups are alkyl groups, they contain one to about four carbon atoms; the alkenyl groups contain two to about six carbon atoms, the aryl groups contain six to about 12 carbon atoms, and the ar-alkyl groups contain seven to about 14 carbon atoms. The alkyl groups include methyl, ethyl, n-propyl, 2-propyl, cyclopropyl, n-butyl, and 2-butyl; the alkenyl groups include ethenyl, propenyl, butenyl, pentenyl, cyclopentenyl, hexenyl, and cyclohexenyl; the aryl groups include phenyl, tolyl, naphthyl, and anthryl groups, and the ar-alkyl groups include benzyl. A brominated noncyclic ether can contain hydrocarbyl groups of different types, such as one or more alkyl groups and one or more aryl groups. Preferred hydrocarbyl groups include alkyl groups, especially methyl and ethyl groups, and aryl groups, especially phenyl groups.

Another way of expressing the brominated noncyclic ethers is with the formula R(OR′)n, where R and R′ are hydrocarbyl groups, and n=1 to 4, preferably 1 or 2. For example, when n=2, the ether has the general formula R(OR¹)(OR²). Each of R and R′ is, independently, an alkyl, alkenyl, or aryl group, with the number of carbon atoms and preferences for each type of hydrocarbyl group as described above.

The bromine content of the noncyclic ether can be on one or more of the hydrocarbyl groups; preferably the one or more bromine atoms are on the same hydrocarbyl group. Preferably, the brominated noncyclic ether contains about 3 to about 10 carbon atoms, more preferably about 3 to about 8 carbon atoms, and preferably has one or two bromine atoms.

The brominated noncyclic ethers usually have a bromine content of about 30 wt % or more, preferably about 30 wt % to about 65 wt %, more preferably about 35 wt % to about 60 wt %, based on the weight of the brominated noncyclic diether. Preferably, the brominated noncyclic ether is 1-bromo-2-methoxyethane, 1-bromo-3-methoxypropane, 2-bromo-1,1-dimethoxyethane, 2-bromo-1,4-dimethoxybenzene, 1-bromo-2-(methoxymethoxy)ethane, 1-bromo-2-ethoxy ethylene (1-bromovinyl ethyl ether), 1,2-dibromo-3-methoxy-1-propene, 1,2-dibromo-3-ethoxy-1-propene, or di(ethylene glycol) dibromovinyl ether.

In other embodiments, the oxygen-containing brominated flame retardant is a brominated cyclic diether. In the brominated cyclic diether, the oxygen atoms are part of the ring structure. In the brominated cyclic diether, the ring is preferably a saturated 5-membered or 6-membered ring, the brominated cyclic diether contains at least one bromine atom, and optionally there is at least one hydrocarbyl group bound to at least one carbon atom of the diether ring. The hydrocarbyl groups bound to one or more carbon atoms of the diether ring are typically saturated hydrocarbyl groups having one to about four carbon atoms, such as methyl, ethyl, n-propyl, 2-propyl, n-butyl, and isobutyl; preferred groups are methyl and ethyl; more preferred hydrocarbyl groups are methyl groups.

Preferably, the brominated cyclic diethers have about three to about ten carbon atoms, more preferably about three to about eight carbon atoms, in the molecule and preferably have one to about 5, more preferably about one to about three, bromine atoms in the molecule. The brominated cyclic diethers normally have a bromine content of about 35 wt % or more, preferably about 35 wt % to about 80 wt %, more preferably about 40 wt % to about 75 wt %, based on the weight of the brominated cyclic diether.

In the brominated cyclic diether, the bromine atoms may be bound to ring carbon atoms, and/or, when present, to at least one hydrocarbyl group bound to a carbon atom of the cyclic diether ring preferably, all of the bromine atoms are in one or more hydrocarbyl groups, or all of the bromine atoms are bound to ring carbon atoms. There may be more than one hydrocarbyl group bound to the ring of the brominated cyclic diether; when there are two or more bromine atoms in the brominated cyclic diether, and there are two or more hydrocarbyl groups bound to the diether ring, the bromine atoms may be in the same or different hydrocarbyl groups, and preferably are in different hydrocarbyl groups.

Preferably, the brominated cyclic diether is 4-bromo-1,3-dioxolane, 2-bromomethyl-1,3-dioxolane, 2-dibromomethyl-1,3-dioxolane, 2-tribromomethyl-1,3-dioxolane, 2,2-bis(bromomethyl)-1,3-dioxolane, 2-(bromomethyl)-1,4-dioxane, 5,5-bis(bromomethyl)-2-methyl-1,3-dioxane, or 5,5-bis(bromomethyl)-2-ethyl-1,3-dioxane.

In still other embodiments, the oxygen-containing brominated flame retardant is a brominated noncyclic carbonate having two hydrocarbyl groups in which at least one hydrocarbyl group has at least one unsaturated carbon-carbon bond, or has aromatic character. At least one hydrocarbyl group of the brominated noncyclic carbonate contains at least one bromine atom. These brominated noncyclic carbonates have a bromine content of about 40 wt % or more, preferably about 40 wt % to about 80 wt %, more preferably about 45 wt % to about 75 wt %, based on the weight of the brominated noncyclic carbonate. In some preferred embodiments, the brominated noncyclic carbonate has about 4 to about 8 carbon atoms in the molecule, and the brominated noncyclic carbonates preferably have one to about four bromine atoms in the molecule.

In the brominated noncyclic carbonates, when the hydrocarbyl groups are alkyl groups, they contain one to about four carbon atoms, the alkenyl groups contain two to about six carbon atoms, the aryl groups contain six to about 12 carbon atoms, and the ar-alkyl groups contain seven to about 14 carbon atoms. The alkyl groups include methyl, ethyl, n-propyl, 2-propyl, n-butyl, and isobutyl; the alkenyl groups include ethenyl, propenyl, butenyl, pentenyl, cyclopentenyl, hexenyl, and cyclohexenyl; the aryl groups include phenyl, tolyl, naphthyl, and anthryl groups, and the ar-alkyl groups include benzyl. A brominated noncyclic carbonate can contain hydrocarbyl groups of different types, such as one alkyl group and one aryl group. Preferred hydrocarbyl groups include alkenyl groups, especially ethenyl and propenyl groups, and aryl groups, especially phenyl groups.

Preferably, the hydrocarbyl groups in the brominated noncyclic carbonate are alkyl, alkenyl, aryl, and/or ar-alkyl groups; more preferably, the hydrocarbyl groups are methyl, ethyl, ethenyl, propenyl, or phenyl groups. In some preferred brominated noncyclic carbonates, one of the hydrocarbyl groups is a methyl group, and the other hydrocarbyl group has at least one unsaturated carbon-carbon bond, or has aromatic character. When there are two or more bromine atoms in the molecule, they can be present in one or both hydrocarbyl groups; when one of the hydrocarbyl groups is a methyl group, the bromine atoms are preferably present on the other hydrocarbyl group. Preferably, the brominated noncyclic carbonate is 3-bromo-2-propenyl methyl carbonate, 2,3-dibromo-2-propenyl methyl carbonate, 2,3,3-tribromo-2-propenyl methyl carbonate, 3-bromo-2-propenyl ethyl carbonate, 2,4-dibromophenyl methyl carbonate, or bis(2,3-dibromo-2-propenyl) carbonate.

In yet other embodiments, the oxygen-containing brominated flame retardant is a brominated cyclic carbonate. In the brominated cyclic carbonate, the carbonate group is part of the ring structure. One or more of the carbon-carbon bonds in the ring of the brominated cyclic carbonate is unsaturated; preferably there is only one unsaturated carbon-carbon-bond in the carbonate ring. In the brominated cyclic carbonates, the carbonate ring is preferably an unsaturated 5-membered or 6-membered ring, the brominated cyclic carbonate contains at least one bromine atom, and optionally at least one hydrocarbyl group is bound to at least one carbon atom of the carbonate ring. The hydrocarbyl groups bound to one or more carbon atoms of the carbonate ring are typically saturated hydrocarbyl groups having one to about four carbon atoms, such as methyl, ethyl, n-propyl, 2-propyl, n-butyl, and isobutyl; preferred groups are methyl and ethyl; more preferred hydrocarbyl groups are methyl groups.

Preferably, the brominated cyclic carbonates have about three to about ten carbon atoms, more preferably about three to about six carbon atoms, in the molecule and preferably have one to about five, more preferably about one to about three, bromine atoms in the molecule. The brominated cyclic carbonates normally have a bromine content of 40 wt % or more, preferably about 40 wt % to about 80 wt %, more preferably about 40 wt % to about 75 wt %, still more preferably about 40 wt % to about 70 wt %, based on the weight of the brominated cyclic carbonate.

In the brominated cyclic carbonate, the bromine atoms may be bound to ring carbon atoms, and/or, when present, to at least one hydrocarbyl group bound to a carbon atom of the cyclic carbonate ring; preferably, all of the bromine atoms are in one or more hydrocarbyl groups, or all of the bromine atoms are bound to ring carbon atoms. There may be more than one hydrocarbyl group bound to the ring of the brominated cyclic carbonate; when there are two or more bromine atoms in the brominated cyclic carbonate, and there are two or more hydrocarbyl groups bound to the carbonate ring, the bromine atoms may be in the same or different hydrocarbyl groups, and preferably are in different hydrocarbyl groups.

Preferably, the brominated cyclic carbonate is 4-bromo-1,3-dioxol-2-one, 4,5-dibromo-1,3-dioxol-2-one, 4-bromomethyl-1,3-dioxol-2-one, 4,4-bis(bromomethyl)-1,3-dioxol-2-one, or 4,5-bis(bromomethyl)-1,3-dioxol-2-one.

In another embodiment, the oxygen-containing brominated flame retardant is 2,4-dibromophenyl methyl carbonate or 2,3-dibromo-2-propenyl methyl carbonate, preferably in an amount of about 10 wt % or more, more preferably about 11 wt % or more, bromine (atoms) relative to the total weight of the solution. Preferably, the liquid electrolyte medium is ethylene carbonate, ethyl methyl carbonate, or a mixture thereof. More preferably, the lithium-containing salt is lithium hexafluorophosphate, lithium di(fluoro)(oxolato)borate, or lithium bis(oxolato)borate.

In some embodiments of the invention, at least one electrochemical additive is included in the nonaqueous electrolyte solution.

In the practice of this invention, the electrochemical additives are soluble in, or miscible with, the liquid medium of the nonaqueous electrolyte solution. Electrochemical additives that are in liquid form are miscible with the liquid medium of the nonaqueous electrolyte solution, where “miscible” means that the electrochemical additives do not form a separate phase from the electrolyte solution. More specifically, an electrochemical additive is miscible if it forms a single phase in a mixture of 30 wt % ethylene carbonate and 70 wt % ethyl methyl carbonate which contains 1.2 M lithium hexafluorophosphate, after 24 hours of shaking in a mechanical shaker, and no separate phase is formed after the shaking is stopped, and the electrochemical additive does not precipitate from, or form a suspension or slurry in, the nonaqueous electrolyte solution.

The term “soluble,” usually used for electrochemical additives in solid form, indicates that, once dissolved, the electrochemical additive does not precipitate from, or form a suspension or slurry in, the nonaqueous electrolyte solution. More specifically, an electrochemical additive is soluble if it dissolves in a mixture of 30 wt % ethylene carbonate and 70 wt % ethyl methyl carbonate which contains 1.2 M lithium hexafluorophosphate, after 24 hours of shaking in a mechanical shaker, if no precipitate, suspension, or slurry is formed after the shaking is stopped. It is recommended and preferred that the electrochemical additive does not cause the precipitation of, or formation of a suspension or slurry of, any of the other components of the nonaqueous electrolyte solution.

The brominated flame retardant, electrochemical additive, and mixtures thereof are generally stable to electrochemical cycling, and preferably have low viscosities and/or do not significantly increase the viscosity of the nonaqueous electrolyte solution.

In various embodiments, the electrochemical additive is selected from a) unsaturated cyclic carbonates containing three to about four carbon atoms, b) fluorine-containing saturated cyclic carbonates containing three to about four carbon atoms and one to about two fluorine atoms, c) tris(trihydrocarbylsilyl) phosphites containing three to about six carbon atoms, d) trihydrocarbyl phosphates containing three to about nine carbon atoms, e) cyclic sultones containing three to about four carbon atoms, f) saturated cyclic hydrocarbyl sulfites having a 5-membered ring and containing two to about four carbon atoms, g) saturated cyclic hydrocarbyl sulfates having a 5-membered ring and containing two to about four carbon atoms, h) cyclic dioxadithio polyoxide compounds having a 6-membered or 7-membered ring and containing two to about four carbon atoms, i) another lithium-containing salt, and j) mixtures of any two or more of the foregoing.

In other embodiments, the electrochemical additive is selected from a) an unsaturated cyclic carbonate in an amount of about 0.5 wt % to about 12 wt %, relative to the total weight of the nonaqueous electrolyte solution, b) a fluorine-containing saturated cyclic carbonate in an amount of about 0.5 wt % to about 8 wt %, relative to the total weight of the nonaqueous electrolyte solution, c) a tris(trihydrocarbylsilyl) phosphite in an amount of about 0.1 wt % to about 5 wt %, relative to the total weight of the nonaqueous electrolyte solution, d) a trihydrocarbyl phosphate in an amount of about 0.5 wt % to about 5 wt %, relative to the total weight of the nonaqueous electrolyte solution, e) a cyclic sultone in an amount of about 0.25 wt % to about 5 wt %, relative to the total weight of the nonaqueous electrolyte solution, f) a saturated cyclic hydrocarbyl sulfite in an amount of about 0.5 wt % to about 5 wt %, relative to the total weight of the nonaqueous electrolyte solution, g) a saturated cyclic hydrocarbyl sulfate in an amount of about 0.25 wt % to about 5 wt %, relative to the total weight of the nonaqueous electrolyte solution, h) a cyclic dioxadithio polyoxide compound in an amount of about 0.5 wt % to about 5 wt %, relative to the total weight of the nonaqueous electrolyte solution, i) another lithium-containing salt in an amount of about 0.5 wt % to about 5 wt %, relative to the total weight of the nonaqueous electrolyte solution, and j) mixtures of any two or more of the foregoing.

In some embodiments, the electrochemical additive is an unsaturated cyclic carbonate containing three to about six carbon atoms, preferably three to about four carbon atoms. Suitable unsaturated cyclic carbonates include vinylene carbonate (1,3-dioxol-2-one), 4-methyl-1,3-dioxol-2-one, and 4,5-dimethyl-1,3-dioxol-2-one; vinylene carbonate is a preferred unsaturated cyclic carbonate. The unsaturated cyclic carbonate is preferably in an amount of about 0.5 wt % to about 12 wt %, more preferably about 0.5 wt % to about 3 wt % or about 8 wt % to about 11 wt %, relative to the total weight of the nonaqueous electrolyte solution.

When the electrochemical additive is a fluorine-containing saturated cyclic carbonate containing three to about five carbon atoms, preferably three to about four carbon atoms, and one to about four fluorine atoms, preferably one to about two fluorine atoms, suitable fluorine-containing saturated cyclic carbonates include 4-fluoro-ethylene carbonate and 4,5-difluoro-ethylene carbonate. Preferably the fluorine-containing saturated cyclic carbonate is 4-fluoro-ethylene carbonate. The fluorine-containing saturated cyclic carbonate is preferably in an amount of about 0.5 wt % to about 8 wt %, more preferably about 1.5 wt % to about 5 wt %, relative to the total weight of the nonaqueous electrolyte solution.

The tris(trihydrocarbylsilyl) phosphite electrochemical additives contain three to about nine carbon atoms, preferably about three to about six carbon atoms; the trihydrocarbylsilyl groups may be the same or different. Suitable tris(trihydrocarbylsilyl) phosphites include tris(trimethylsilyl) phosphite, bis(trimethylsilyl)(triethylsilyl) phosphite, tris(triethylsilyl) phosphite, bis(trimethylsilyl)(triethylsilyl) phosphite, bis(trimethylsilyl)(tri-n-propylsilyl)phosphite, and tris(tri-n-propylsilyl) phosphite; tris(trimethylsilyl) phosphite is a preferred tris(trihydrocarbylsilyl) phosphite. The tris(trihydrocarbylsilyl) phosphite is preferably in an amount of about 0.1 wt % to about 5 wt %, more preferably about 0.15 wt % to about 4 wt %, even more preferably about 0.2 wt % to about 3 wt %, relative to the total weight of the nonaqueous electrolyte solution.

In some embodiments, the electrochemical additive is a trihydrocarbyl phosphate containing three to about twelve carbon atoms, preferably three to about nine carbon atoms. The hydrocarbyl groups can be saturated or unsaturated, and the hydrocarbyl groups in the trihydrocarbyl phosphate may be the same or different. Suitable trihydrocarbyl phosphates include trimethyl phosphate, triethyl phosphate, dimethyl ethyl phosphate, tri-n-propyl phosphate, triallyl phosphate, and trivinyl phosphate; triallyl phosphate is a preferred trihydrocarbyl phosphate. The trihydrocarbyl phosphate is usually in an amount of about 0.5 wt % to about 5 wt %, preferably about 1 wt % to about 5 wt %, more preferably about 2 wt % to about 4 wt %, relative to the total weight of the nonaqueous electrolyte solution.

When the electrochemical additive is a cyclic sultone containing three to about eight carbon atoms, preferably three to about four carbon atoms, suitable cyclic sultones include 1-propane-1,3-sultone (1,3-propane sultone), 1-propene-1,2-sultone (1,3-propene sultone), 1,3-butane sultone (5-methyl-1,2-oxathiolane 2,2-dioxide), 2,4-butane sultone (3-methyl-1,2-oxathiolane 2,2-dioxide), 1,4-butane sultone (1,2-oxathiane 2,2-dioxide), 2-hydroxy-alpha-toluenesulfonic acid sultone (3H-1,2-benzoxathiole 2,2-dioxide), and 1,8-naphthosultone; preferred cyclic sultones include 1-propane-1,3-sultone and 1-propene-1,3-sultone. The cyclic sultone is preferably in an amount of about 0.25 wt % to about 5 wt %, more preferably about 0.5 wt % to about 4 wt %, relative to the total weight of the nonaqueous electrolyte solution.

The saturated cyclic hydrocarbyl sulfite electrochemical additive contains two to about six carbon atoms, preferably two to about four carbon atoms, and has a 5-membered or 6-membered ring, preferably a 5-membered ring. One or more substituents can be present on the ring, such as methyl or ethyl groups, preferably one or more methyl groups, more preferably, no sub stituents are present on the ring. Suitable saturated cyclic hydrocarbyl sulfites include 1,3,2-dioxathiolane, 2-oxide (1,2-ethylene sulfite), 1,2-propanediol sulfite (1,2-propylene sulfite), 4,5-dimethyl-1,3,2-dioxathiolane 2-oxide, 1,3,2-dioxathiane 2-oxide, 4-methyl-1,3-dioxathiane, 2-oxide (1,3-butylene sulfite); preferred cyclic hydrocarbyl sulfites include 1,3,2-dioxathiolane, 2-oxide (1,2-ethylene sulfite). The cyclic hydrocarbyl sulfite is preferably in an amount of about 0.5 wt % to about 5 wt %, more preferably about 1 wt % to about 4 wt %, relative to the total weight of the nonaqueous electrolyte solution.

In some embodiments, the electrochemical additive is a saturated cyclic hydrocarbyl sulfate containing two to about six carbon atoms, preferably two to about four carbon atoms, and has a 5-membered or 6-membered ring, preferably a 5-membered ring. One or more substituents can be present on the ring, such as methyl or ethyl groups, preferably one or more methyl groups, more preferably, no substituents are present on the ring. Suitable saturated cyclic hydrocarbyl sulfates include 1,3,2-dioxathiolane 2,2-dioxide (1,2-ethylene sulfate), 1,3,2-dioxathiane 2,2-dioxide (1,3-propylene sulfate), 4-methyl-1,3,2-dioxathiane 2,2-dioxide (1,3-butylene sulfate), and 5,5-dimethyl-1,3,2-dioxathiane 2,2-dioxide. The saturated cyclic hydrocarbyl sulfate is preferably in an amount of about 0.25 wt % to about 5 wt %, more preferably about 1 wt % to about 4 wt %, relative to the total weight of the nonaqueous electrolyte solution.

When the electrochemical additive is a cyclic dioxadithio polyoxide compound, the cyclic dioxadithio polyoxide compound contains two to about six carbon atoms, preferably two to about four carbon atoms, and has 6-membered, 7-membered, or 8-membered ring. Preferably, the cyclic dioxadithio polyoxide compound contains two to about four carbon atoms, and has 6-membered or 7-membered ring. One or more substituents can be present on the ring, such as methyl or ethyl groups, preferably one or more methyl groups, more preferably, no substituents are present on the ring. Suitable cyclic dioxadithio polyoxide compounds include 1,5,2,4-dioxadithiane 2,2,4,4-tetroxide, 1,5,2,4-dioxadithiepane 2,2,4,4-tetraoxide (cyclodisone), 3-methyl-1,5,2,4-dioxadithiepane, 2,2,4,4-tetraoxide, and 1,5,2,4-dioxadithiocane, 2,2,4,4-tetraoxide; 1,5,2,4-dioxadithiane 2,2,4,4-tetroxide is preferred. The cyclic dioxadithio polyoxide compound is preferably in an amount of about 0.5 wt % to about 5 wt %, more preferably about 1 wt % to about 4 wt %, relative to the total weight of the nonaqueous electrolyte solution.

The phrases “another lithium-containing salt” and “other lithium containing salt” indicate that there are at least two lithium salts used in the preparation of the electrolyte solution. When the electrochemical additive is another lithium-containing salt, it is preferably in an amount of about 0.5 wt % to about 5 wt % relative to the total weight of the nonaqueous electrolyte solution. Suitable lithium-containing salts include all of the lithium-containing salts listed above; lithium di(fluoro)(oxolato)borate and lithium bis(oxolato)borate are preferred.

Mixtures of any two or more of the foregoing electrochemical additives can be used, including different electrochemical additives of the same type and/or electrochemical additives of different types. When mixtures of electrochemical additives are used, the combined amount of the electrochemical additives is about 0.25 wt % to about 5 wt % relative to the total weight of the nonaqueous electrolyte solution. Mixtures of an unsaturated cyclic carbonate and a saturated cyclic hydrocarbyl sulfite or mixtures of a cyclic sultone, a tris(trihydrocarbylsilyl) phosphite, and a cyclic dioxadithio polyoxide compound are preferred.

Preferred types of electrochemical additives include saturated cyclic hydrocarbyl sulfates, cyclic sultones, tris(trihydrocarbylsilyl) phosphites, and another lithium-containing salt, especially when not used with other electrochemical additives. More preferably, the saturated cyclic hydrocarbyl sulfate is in an amount of about 1 wt % to about 4 wt %, the cyclic sultone is in an amount of about 0.5 wt % to about 4 wt %, the tris(trihydrocarbylsilyl) phosphite is in an amount of about 0.2 wt % to about 3 wt %, and another lithium-containing salt is in an amount of about 1 wt % to about 4 wt %, each relative to the total weight of the nonaqueous electrolyte solution.

In other embodiments, the electrochemical additive is selected from vinylene carbonate, 4-fluoro-ethylene carbonate, tris(trimethylsilyl)phosphite, triallyl phosphate, 1-propane-1,3-sultone, 1-propene-1,3-sultone, ethylene sulfite, 1,3,2-dioxathiolane 2,2-dioxide, 1,5,2,4-dioxadithiane 2,2,4,4-tetroxide, lithium di(fluoro)(oxolato)borate, lithium bis(oxolato)borate, lithium hexafluorophosphate, and mixtures of any two or more of these. The electrochemical additive is preferably 1,3,2-dioxathiolane 2,2-dioxide, 1-propane-1,3-sultone, 1-propene-1,3-sultone, tris(trimethylsilyl)phosphite, lithium di(fluoro)(oxolato)borate, or lithium bis(oxolato)borate, more preferably 1,3,2-dioxathiolane 2,2-dioxide, 1-propene-1,3-sultone, or lithium bis(oxolato)borate. More preferred electrochemical additives are 1,3,2-dioxathiolane 2,2-dioxide and lithium bis(oxolato)borate. Amounts and preferences therefor are as described above.

Mixtures of any two or more of the foregoing electrochemical additives can be used. When mixtures of electrochemical additives are used, the combined amount of the electrochemical additives is about 0.25 wt % to about 5 wt %, relative to the total weight of the nonaqueous electrolyte solution.

Additional ingredients that are often included in electrolyte solutions for lithium batteries can also be present in the electrolyte solutions of the present invention. Such additional ingredients include succinonitrile and silazane compounds such as hexamethyldisilazane. Typically, the amount of an optional ingredient is in the range of about 1 wt % to about 5 wt %, preferably about 2 wt % to about 4 wt %, relative to the total weight of the nonaqueous electrolyte solution.

Another embodiment of this invention provides a process for producing a nonaqueous electrolyte solution for a lithium battery. The process comprises combining components comprising i) a liquid electrolyte medium; ii) a lithium-containing salt; and iii) at least one oxygen-containing brominated flame retardant. Optionally, the components further comprise iv) at least one electrochemical additive as described above. The oxygen-containing brominated flame retardant is present in the electrolyte solution in a flame retardant amount. The ingredients can be combined in any order, although it is preferable to add all of the components to the liquid electrolyte medium. Optional ingredients are also preferably added to the liquid electrolyte medium. Features of, and preferences for, the liquid electrolyte medium, lithium-containing salt, oxygen-containing brominated flame retardant(s), electrochemical additive(s), and amounts of each component, are as described above.

Still another embodiment of this invention provides a process for producing a nonaqueous electrolyte solution for a lithium battery. The process comprises combining components comprising i) a liquid electrolyte medium; ii) a lithium-containing salt; and iii) at least one brominated flame retardant. Optionally, the components further comprise iv) at least one electrochemical additive as described above. The brominated flame retardant is selected from the group consisting of 1-bromo-2-methoxyethane, 1-bromo-3-methoxypropane, 2-bromo-1,1-dimethoxyethane, 1-bromo-2-(methoxymethoxy)ethane, 1-bromovinyl ethyl ether, 1,2-dibromo-3-methoxy-1-propene, 1,2-dibromo-3-ethoxy-1-propene, di(ethylene glycol) dibromovinyl ether, 4-bromo-1,3-dioxolane, 2-bromomethyl-1,3-dioxolane, 2-dibromomethyl-1,3-dioxolane, 2-tribromomethyl-1,3-dioxolane, 2,2-bis(bromomethyl)-1,3-dioxolane, 2-(bromomethyl)-1,4-dioxane, 5,5-bis(bromomethyl)-2-methyl-1,3-dioxane, 5,5-bis(bromomethyl)-2-ethyl-1,3-dioxane, 3-bromo-2-propenyl methyl carbonate, 2,3-dibromo-2-propenyl methyl carbonate, 2,3,3-tribromo-2-propenyl methyl carbonate, 3-bromo-2-propenyl ethyl carbonate, 2,4-dibromophenyl methyl carbonate, bis(2,3-dibromo-2-propenyl) carbonate, 4-bromo-1,3-dioxol-2-one, 4,5-dibromo-1,3-dioxol-2-one, 4-bromomethyl-1,3-dioxol-2-one, 4,4-bis(bromomethyl)-1,3-dioxol-2-one, and 4,5-bis(bromomethyl)-1,3-dioxol-2-one. Preferences for the liquid electrolyte medium, lithium-containing salt, electrochemical additive(s), and amounts of each component, are as described above.

The nonaqueous electrolyte solutions of the present invention, which contain one or more brominated flame retardants, are typically used in nonaqueous lithium batteries comprising a positive electrode, a negative electrode, and the nonaqueous electrolyte solution. A nonaqueous lithium battery can be obtained by injecting a nonaqueous electrolyte solution between the negative electrode and the positive electrode optionally having a separator therebetween.

The molecules 1,2-dibromo-3-ethoxy-1-propene, di(ethylene glycol) dibromovinyl ether, 3-bromo-2-propenyl methyl carbonate, 2,3-dibromo-2-propenyl methyl carbonate, 2,3,3-tribromo-2-propenyl methyl carbonate, 3-bromo-2-propenyl ethyl carbonate, 2,4-dibromophenyl methyl carbonate, 5,5-bis(bromomethyl)-2-methyl-1,3-dioxane, 5,5-bis(bromomethyl)-2-ethyl-1,3-dioxane, and 4-bromo-1,3-dioxolane are new compositions of matter.

The following examples are presented for purposes of illustration, and are not intended to impose limitations on the scope of this invention.

In Example 1, a modified horizontal UL-94 test was performed. This modified horizontal UL-94 test is quite similar to known, published horizontal UL-94 tests. See in this regard, e.g., Otsuki, M. et al. “Flame-Retardant Additives for Lithium-Ion Batteries.” Lithium-Ion Batteries. Ed. M. Yoshio et al. New York, Springer, 2009, 275-289. The modified UL-94 test was as follows:
Wicks were cut from round fiberglass wick, and cut edges were made smooth, and then dust and particles were removed from the wick surface, The wicks were dried for 20 hours at 120° C. prior to testing. Wicks were 5±0.1 inch (12.7±0.25 cm) long. Each specimen to be tested was prepared in a dry box in a 4 oz. (120 mL) glass jar, by combining the desired amount of flame retardant and, when present, electrochemical additive, with the desired amount of the electrolyte solution, e.g., 20 wt % of the brominated flame retardant and 80 wt % of the electrolyte solution were combined to form the electrolyte solution containing the flame retardant. Prior to combination with the flame retardant, the electrolyte solution contained 1.2 M LiPF₆ in ethylene carbonate/ethyl methyl carbonate (wt ratio 3:7). Each wick was soaked in the electrolyte solution for 30 minutes.
Each specimen was removed from the electrolyte solution and held over the electrolyte solution until no dripping occurred, and then placed in a 4 oz. (120 mL) glass jar; the cap was closed to prevent electrolyte solution from evaporating.
The burner was ignited and adjusted to produce a blue flame 20±1 mm high.
A specimen was removed from its 4 oz. (120 mL) glass jar, and the specimen was placed on a metal support fixture in a horizontal position, secured at one end of the wick.
If an exhaust fan was running, it was shut off for the test.
The flame was at an angle of 45±2 degrees to the horizontal wick. One way to accomplish this when the burner had a burner tube was to incline the central axis of the burner tube toward an end of the specimen at an angle of 45±2 degrees from the horizontal.
The flame was applied to the free end of the specimen for 30±1 seconds without changing its position; the burner was removed after 30±1 seconds, or as soon as the combustion front on the specimen reached the 1 inch (2.54 cm) mark.
If the specimen continued to burn after removal of the test flame, the time in seconds was recorded, for either the flame to extinguish or for the combustion front (flame) to travel from the 1 inch (2.54 cm) mark to the 4 inch (10.16 cm) mark.

A specimen was considered to be “not flammable” if the flame extinguished when the burner was removed. A specimen was considered to be “flame retardant” if the flame extinguished before reaching the 1 inch (2.54 cm) mark. A specimen was considered to be “self-extinguishing” if the flame went out before reaching the 4 inch (10.16 cm) mark.

Each modified horizontal UL-94 test result reported below is the average of three runs.

### EXAMPLE 1

Various nonaqueous electrolyte solutions containing different oxygen-containing brominated flame retardants, prepared as described above, were subjected to the modified UL-94 test described above. Results are summarized in Table 1 below; as noted above, the reported numbers are an average value from three runs.

TABLE 1

| | Flame | | | |
|---|---|---|---|---|
| | retardant | Bromine | | |
| | wt % | wt % | | Time to |
| Flame retardant | in soln. | in soln. | Result | extinguish |
| 2,4-dibromophenyl methyl carbonate | 30 | 15.6 | flame retardant | 22 s |
| 2,3-dibromo-2-propenyl methyl carbonate | 20 | 11.7 | flame retardant | 36 s |
| 2-bromomethyl-1,3-dioxolane | 20 | 9.6 | self-extinguish. | 62 s |
| 1-bromo-2-methoxyethane | 30 | 17.2 | flame retardant | 22 s |
| 2-bromo-1,1-dimethoxyethane | 30 | 14.2 | flame retardant | 12 s |
| 1-bromo-2-(methoxymethoxy)ethane | 30 | 14.2 | flame retardant | 18 s |
| 3-bromo-2-propenyl methyl carbonate | 25 | 10.26 | flame retardant | 26 s |

### EXAMPLE 2

Tests of some flame retardants in coin cells were also carried out. Coin cells were assembled using nonaqueous electrolyte solutions containing the desired amount of flame retardant. The coin cells were then subjected to electrochemical cycling of CCCV charging to 4.2 V at C/5, with a current cutoff of C/50 in the CV portion, and CC discharge at C/5 to 3.0 V.

One sample was a nonaqueous electrolyte solution without a flame retardant, and contained 1.2 M LiPF₆ in ethylene carbonate/ethyl methyl carbonate (wt ratio 3:7). The rest of the samples contained the desired amount of flame retardant in the electrolyte solution; some solutions also contained an additive in addition to the flame retardant. Results are summarized in Table 2 below; the error range in the Coulombic efficiencies is about 0.5% to about ±1.0%.

TABLE 2

| | Flame | | | | |
|---|---|---|---|---|---|
| | retardant | Bromine | | Coulombic efficiency | |
| Chemical Name | in soln. | in soln. | | 1st cycle | 10th cycle |
| Electrolyte soln.¹ | 0 | 0 | | 81.8% | 99.6% |
| 2-bromo-1,1-dimethoxyethane² | 8 wt % | 3.79 | wt % | 37.6% | 79.5% |
| 1-bromovinyl ethyl ether² | 8 wt % | 4.24 | wt % | 29.9% | 80.7% |
| 1-bromo-2-(methoxymethoxy)ethane² | 8 wt % | 3.79 | wt % | 34.2% | 91.9% |
| 2,4-dibromophenyl methyl carbonate³ | 8 wt % | 4.2 | wt % | 46.6% | 92.5% |
| 4-bromo-1,3-dioxol-2-one³ | 8 wt % | 3.9 | wt % | 60.5% | 89.6% |
| 2-bromo-1,4-dimethoxybenzene³ | 8 wt % | 3 | wt % | 0.4% | 38.1% |
| 2-bromomethyl-1,3-dioxolane³ | 8 wt % | 3.8 | wt % | 31.8% | 84.0% |
| 3-bromo-2-propenyl methyl carbonate² | 8 wt % | 3.28 | wt % | 70.0% | 98.9% |

¹Comparative run.
²Data is from single best-performing cell.
³Data is an average from multiple cells (“multiple cells” usually menas two or three cells).

### EXAMPLE 3

### Synthesis of 1,2-dibromo-3-ethoxy-1-propene

Aqueous NaOH (50 wt %, 17.5 g), 2,3-dibromoallyl alcohol (21.6 g, 0.1 mol), tetrabutylammonium bromide (1.0 g), and bromoethane (21.8 g, 0.2 mol) were charged to a 500-mL jacketed round bottom flask and this mixture was stirred while heating to 40° C., and then heated at 40° C. for 6 hours. After cooling the mixture to room temperature, the reaction mass was diluted with ethyl ether (120 mL) and washed with deionized water (100 mL). After drying over MgSO₄ and then filtering to remove solids, the solvent was removed on a rotary evaporator. The product was further dried under high vacuum to give 1,2-dibromo-3-ethoxy-1-propene as a clear liquid (19.54 g; 79% yield).

### EXAMPLE 4

### Synthesis of di(ethylene glycol) dibromovinyl ether

Dichloromethane (100 mL) and di(ethylene glycol) divinyl ether (31.6 g, 0.2 mol) were introduced to a 250-mL round bottom flask and then magnetically stirred in ice cold water. an ice cold water bath. To this mixture Br₂ (64 g, 0.4 mol) was added to the flask slowly using a peristaltic pump. After all of the Br₂ had been added, the reaction mixture was stirred for 2 hours while allowing the reaction mixture to reach room temperature. The reaction flask was then was placed in an ice cold water bath, and triethylamine (50 g, 0.494 mol) was added to the flask dropwise from an addition funnel. After all of the triethylamine had been added, the reaction mixture was stirred for 4 hours while allowing the reaction mixture to reach room temperature. The mixture was filtered to remove the solid that had formed, and the residual solution was collected in a 250-mL round flask. The solvent was removed from residual solution in the round flask, and then the residual liquid in the round flask was passed through a silica gel column to obtain di(ethylene glycol) dibromovinyl ether (35.5 g; 56.2% yield).

### EXAMPLE 5

### Synthesis of 5,5-bis(bromomethyl)-2-methyl-1,3-dioxane

Toluene (100 mL), p-toluenesulphonic acid monohydrate (0.5 g), and 2,2-bis(bromomethyl)-1,3-propanediol (52.4 g, 0.2 mol) were introduced to a 250-mL round bottom flask and then magnetically stirred at room temperature. To this mixture acetaldehyde (12 g, 0.27 mol) was added. The reaction mixture was heated to reflux, kept at reflux for 2 hours, and then cooled to room temperature and washed with dilute aqueous NaOH (30 mL) and followed by water (30 mL). The mixture was allowed to separate into phases; the organic phase was further treated. The solvent was removed from the organic phase, and then the residual liquid from the organic phase was purified by vacuum distillation to obtain 5,5-bis(bromomethyl)-2-methyl-1,3-dioxane (54 g; 99% yield).

### EXAMPLE 6

### Synthesis of 5,5-bis(bromomethyl)-2-ethyl-1,3-dioxane

Toluene (150 mL), p-toluenesulphonic acid monohydrate (1.0 g), and 2,2-bis(bromomethyl)-1,3-propanediol (104.8 g, 0.4 mol) were introduced to a 250-mL round bottom flask and then magnetically stirred at room temperature. To this mixture propionaldehyde (29.0 g, 0.5 mol) was added. The reaction mixture was heated to reflux, kept at reflux for 2 hours, and then cooled to room temperature and washed with dilute aqueous NaOH (200 mL) and followed by water (100 mL). The mixture was allowed to separate into phases; the organic phase was further treated. The solvent was removed from the organic phase, and then the residual liquid from the organic phase was purified by vacuum distillation to obtain 5,5-bis(bromomethyl)-2-ethyl-1,3-dioxane (120.8 g; 99% yield).

### EXAMPLE 7

### Synthesis of 3-bromo-2-propenyl methyl carbonate (bromoallyl methyl carbonate)

Dichloromethane (150 mL) and allyl methyl carbonate (34.8 g, 0.3 mol) were introduced to a 500-mL round bottom flask and then magnetically stirred in an ice cold water bath. To this mixture Br₂ (48 g, 0.3 mol) was added slowly using a peristaltic pump. After all of the Br₂ had been added, the reaction mixture was stirred for 2 hours while allowing the reaction mixture to reach room temperature. The reaction flask was then was placed in an ice cold water bath, and triethylamine (40 g, 0.395 mol) was added to the flask dropwise from an addition funnel. After all of the triethylamine had been added, the reaction mixture was stirred for 4 hours while allowing the reaction mixture to reach room temperature. The mixture was filtered to remove the solid that had formed, and the residual solution was collected in a 500-mL round flask. The solvent was removed from residual solution in the round flask, and then the residual liquid in the round flask was passed through the silica gel column and purified by vacuum distillation to obtain bromoallyl methyl carbonate (32.3 g; 55.2% yield).

### EXAMPLE 8

### Synthesis of 3-bromo-2-propenyl ethyl carbonate (bromoallyl ethyl carbonate)

Dichloromethane (100 mL) and allyl ethyl carbonate (26 g, 0.2 mol) were introduced to a 250-mL round bottom flask and then magnetically stirred in an ice cold water bath. To this mixture Br₂ (32 g, 0.2 mol) was added slowly using a peristaltic pump. After all of the Br₂ had been added, the reaction mixture was stirred for 2 hours while allowing the reaction mixture to reach room temperature. The reaction flask was then was placed in an ice cold water bath, and triethylamine (22.3 g, 0.22 mol) was added to the flask dropwise from an addition funnel. After all of the triethylamine had been added, the reaction mixture was stirred for 4 hours while allowing the reaction mixture to reach room temperature. The mixture was filtered to remove the solid that had formed, and the residual solution was collected in a 250-mL round flask. The solvent was removed from residual solution in the round flask, and then the residual liquid in the round flask was passed through the silica gel column and purified by vacuum distillation to obtain bromoallyl ethyl carbonate (19.7 g; 47% yield).

### EXAMPLE 9

### Synthesis of 2,3-dibromo-2-propenyl methyl carbonate

Dichloromethane (75 g) and 2,3-dibromo-2-propen-1-ol (21.6 g, 0.1 mol) were introduced to a 250-mL round bottom flask and then magnetically stirred in a cold water bath. To this mixture triethylamine (11.3 g, 0.11 mol) was added slowly, and then methyl chloroformate (10.4 g, 0.11 mol) was added dropwise over 1 hour using a syringe pump. After all of the methyl chloroformate had been added, the reaction mixture was stirred for 1 hour while allowing the reaction mixture to reach room temperature and then water was added to quench the reaction. Aqueous HCl (10 wt %) was added to adjust the pH to 1. The mixture was allowed to separate into phases; the organic phase was further treated. The organic phase was washed with water (25 mL), dilute aqueous NaOH (25 mL) and more water (25 mL). The solvent was removed from the organic phase, and then the residual liquid from the organic phase was purified by vacuum distillation to obtain 2,3-dibromo-2-propenyl methyl carbonate (16.4 g; 60% yield).

### EXAMPLE 10

### Synthesis of 2, 4-Dibromophenyl methyl carbonate

Dichloromethane (100 g) and 2,4-dibromophenol (25.2 g, 0.1 mol) were introduced to a 250-mL round bottom flask and then magnetically stirred in a cold water bath. To this mixture triethylamine (11.3 g, 0.11 mol) was added slowly, and then methyl chloroformate (10.4 g, 0.11 mol) was added dropwise over 1 hour using a syringe pump. After all of the methyl chloroformate had been added, the temperature of the reaction mixture was raised to 35° C., and the reaction mixture was stirred at 35° C. for 1 hour, and then water was added to quench the reaction. Aqueous HCl (10 wt %) was added to adjust the pH to 1. The mixture was allowed to separate into phases; the organic phase was further treated. The organic phase was washed with water (25 mL), dilute aqueous NaOH (25 mL) and more water (25 mL). The solvent was removed from the organic phase, and then the residual liquid from the organic phase was purified by vacuum distillation to obtain 2,3-dibromo-2-propenyl methyl carbonate (30.7 g; 99% yield).

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition. Also, even though the claims hereinafter may refer to substances, components and/or ingredients in the present tense (“comprises”, “is”, etc.), the reference is to the substance, component or ingredient as it existed at the time just before it was first contacted, blended or mixed with one or more other substances, components and/or ingredients in accordance with the present disclosure. The fact that a substance, component or ingredient may have lost its original identity through a chemical reaction or transformation during the course of contacting, blending or mixing operations, if conducted in accordance with this disclosure and with ordinary skill of a chemist, is thus of no practical concern.

The invention may comprise, consist, or consist essentially of the materials and/or procedures recited herein.

As used herein, the term “about” modifying the quantity of an ingredient in the compositions of the invention or employed in the methods of the invention refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term about also encompasses amounts that differ due to different equilibrium conditions for a composition resulting from a particular initial mixture. Whether or not modified by the term “about”, the claims include equivalents to the quantities.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

This invention is susceptible to considerable variation in its practice. Therefore the foregoing description is not intended to limit, and should not be construed as limiting, the invention to the particular exemplifications presented hereinabove.

1. A nonaqueous electrolyte solution for a lithium battery, which solution comprises
i) a liquid electrolyte medium;
ii) a lithium-containing salt; and
iii) at least one oxygen-containing brominated flame retardant
A) selected from
a) a brominated noncyclic ether,
b) a brominated cyclic diether,
c) a brominated noncyclic carbonate having two hydrocarbyl groups in which at least one hydrocarbyl group has at least one unsaturated carbon-carbon bond, or has aromatic character, and
d) a brominated cyclic carbonate having a carbonate ring, which carbonate ring has at least one unsaturated carbon-carbon bond; or
B) selected from the group consisting of 1-bromo-2-methoxyethane, 1-bromo-3-methoxypropane, 2-bromo-1,1-dimethoxyethane, 2-bromo-1,4-dimethoxybenzene, 1-bromo-2-(methoxymethoxy)ethane, 1-bromovinyl ethyl ether, 1,2-dibromo-3-methoxy-1-propene, 1,2-dibromo-3-ethoxy-1-propene, di(ethylene glycol) dibromovinyl ether, 4-bromo-1,3-dioxolane, 2-bromomethyl-1,3-dioxolane, 2-dibromomethyl-1,3-dioxolane, 2-tribromomethyl-1,3-dioxolane, 2,2-bis(bromomethyl)-1,3-dioxolane, 2-(bromomethyl)-1,4-dioxane, 5,5-bis(bromomethyl)-2-methyl-1,3-dioxane, 5,5-bis(bromomethyl)-2-ethyl-1,3-dioxane, 3-bromo-2-propenyl methyl carbonate, 2,3-dibromo-2-propenyl methyl carbonate, 2,3,3-tribromo-2-propenyl methyl carbonate, 3-bromo-2-propenyl ethyl carbonate, 2,4-dibromophenyl methyl carbonate, bis(2,3-dibromo-2-propenyl) carbonate, 4-bromo-1,3-dioxol-2-one, 4,5-dibromo-1,3-dioxol-2-one, 4-bromomethyl-1,3-dioxol-2-one, 4,4-bis(bromomethyl)-1,3-dioxol-2-one, and 4,5-bis(bromomethyl)-1,3-dioxol-2-one.

2. A solution as in claim 1 wherein the brominated flame retardant of A) has a boiling point of about 95° C. or higher, or wherein the brominated flame retardant has a boiling point in the range of about 75° C. to about 450° C.

3. (canceled)

4. A solution as in claim 1 wherein the oxygen-containing brominated flame retardant of A)
has about three to about ten carbon atoms, one to about five bromine atoms, and/or a bromine content of about 35 wt % or more relative to the total weight of the oxygen-containing brominated flame retardant, or
is a brominated cyclic diether or a brominated cyclic carbonate which has a 5-membered or 6-membered ring, and optionally has about three to about ten carbon atoms, one to about five bromine atoms, and/or a bromine content of about 35 wt % or more relative to the total weight of the oxygen-containing brominated flame retardant.

5. (canceled)

6. A solution as in claim 1 wherein the oxygen-containing brominated flame retardant of A) is
a brominated noncyclic ether which has about three to about ten carbon atoms and a bromine content of about 30 wt % or more relative to the total weight of the oxygen-containing brominated flame retardant;
a brominated cyclic diether which has about three to about eight carbon atoms and one to about three bromine atoms;
a brominated noncyclic carbonate which has about four to about eight carbon atoms and one to about four bromine atoms; or
a brominated cyclic carbonate which has about three to about ten carbon atoms, one to about five bromine atoms, and a bromine content of about 40 wt % or more relative to the total weight of the oxygen-containing brominated flame retardant.

7. A solution as in claim 1 wherein the oxygen-containing brominated flame retardant of A) is
a brominated cyclic diether containing two or more bromine atoms wherein all of the bromine atoms are in one or more hydrocarbyl groups, or all of the bromine atoms are bound to carbon atoms of the ring;
a brominated noncyclic carbonate which has at least one alkyl, alkenyl, aryl, or ar-alkyl group; or
a brominated cyclic carbonate containing two or more bromine atoms wherein all of the bromine atoms are in one or more hydrocarbyl groups bound to the carbonate ring, or all of the bromine atoms are bound to carbon atoms of the carbonate ring.

8. A solution as in claim 7 wherein the oxygen-containing brominated flame retardant of A) is
a brominated cyclic diether containing two or more bromine atoms and having two or more hydrocarbyl groups bound to the carbonate ring, and wherein the bromine atoms are in different hydrocarbyl groups;
a brominated noncyclic carbonate having hydrocarbyl groups which are selected from methyl, ethyl, propenyl, and phenyl groups; or
a brominated cyclic carbonate containing two or more bromine atoms and having two or more hydrocarbyl groups bound to the carbonate ring, wherein the bromine atoms are in different hydrocarbyl groups.

9. A solution as in claim 1 wherein the oxygen-containing brominated flame retardant of A) is
a brominated cyclic diether having two or more hydrocarbyl groups bound to the carbonate ring, which hydrocarbyl groups are methyl groups; or
a brominated cyclic carbonate having two or more hydrocarbyl groups bound to the carbonate ring, which hydrocarbyl groups are methyl groups; or
a brominated noncyclic carbonate in which one hydrocarbyl group is a methyl group; or 2,4-dibromophenyl methyl carbonate or 2,3-dibromo-2-propenyl methyl carbonate.

10-11. (canceled)

12. A solution as in claim 1 wherein the oxygen-containing brominated flame retardant is in an amount of about 10 wt % or more bromine relative to the total weight of the solution.

13. A solution as in claim 1 wherein the liquid electrolyte medium is ethylene carbonate, ethyl methyl carbonate, or a mixture thereof, and/or wherein the lithium-containing salt is lithium hexafluorophosphate, lithium di(fluoro)(oxolato)borate, or lithium bis(oxolato)borate.

14. A solution as in claim 1 further comprising at least one electrochemical additive selected from:
a) unsaturated cyclic carbonates containing three to about six carbon atoms,
b) fluorine-containing saturated cyclic carbonates containing three to about five carbon atoms and one to about four fluorine atoms,
c) tris(trihydrocarbylsilyl) phosphites containing three to about nine carbon atoms,
d) trihydrocarbyl phosphates containing three to about twelve carbon atoms,
e) cyclic sultones containing three to about eight carbon atoms,
f) saturated cyclic hydrocarbyl sulfites having a 5-membered or 6-membered ring and containing two to about six carbon atoms,
g) saturated cyclic hydrocarbyl sulfates having a 5-membered or 6-membered ring and containing two to about six carbon atoms,
h) cyclic dioxadithio polyoxide compounds having a 6-membered, 7-membered, or 8-membered ring and containing two to about six carbon atoms,
i) another lithium-containing salt, and
j) mixtures of any two or more of the foregoing.

15. A solution as in claim 14 wherein the electrochemical additive is selected from:
a) unsaturated cyclic carbonates containing three to about four carbon atoms,
b) fluorine-containing saturated cyclic carbonates containing three to about four carbon atoms and one to about two fluorine atoms,
c) tris(trihydrocarbylsilyl) phosphites containing three to about six carbon atoms,
d) trihydrocarbyl phosphates containing three to about nine carbon atoms,
e) cyclic sultones containing three to about four carbon atoms,
f) saturated cyclic hydrocarbyl sulfites having a 5-membered ring and containing two to about four carbon atoms,
g) saturated cyclic hydrocarbyl sulfates having a 5-membered ring and containing two to about four carbon atoms,
h) cyclic dioxadithio polyoxide compounds having a 6-membered or 7-membered ring and containing two to about four carbon atoms,
i) another lithium-containing salt, and
j) mixtures of any two or more of the foregoing.

16. A solution as in claim 14 wherein the electrochemical additive is selected from:
a) an unsaturated cyclic carbonate in an amount of about 0.5 wt % to about 12 wt %, relative to the total weight of the nonaqueous electrolyte solution,
b) a fluorine-containing saturated cyclic carbonate in an amount of about 0.5 wt % to about 8 wt %, relative to the total weight of the nonaqueous electrolyte solution,
c) a tris(trihydrocarbylsilyl) phosphite in an amount of about 0.1 wt % to about 5 wt %, relative to the total weight of the nonaqueous electrolyte solution,
d) a trihydrocarbyl phosphate in an amount of about 0.5 wt % to about 5 wt %, relative to the total weight of the nonaqueous electrolyte solution,
e) a cyclic sultone in an amount of about 0.25 wt % to about 5 wt %, relative to the total weight of the nonaqueous electrolyte solution,
f) a saturated cyclic hydrocarbyl sulfite in an amount of about 0.5 wt % to about 5 wt %, relative to the total weight of the nonaqueous electrolyte solution,
g) a saturated cyclic hydrocarbyl sulfate in an amount of about 0.25 wt % to about 5 wt %, relative to the total weight of the nonaqueous electrolyte solution,
h) a cyclic dioxadithio polyoxide compound in an amount of about 0.5 wt % to about 5 wt %, relative to the total weight of the nonaqueous electrolyte solution,
i) another lithium-containing salt in an amount of about 0.5 wt % to about 5 wt %, relative to the total weight of the nonaqueous electrolyte solution, and
j) mixtures of any two or more of the foregoing.

17. A solution as in claim 14 wherein the electrochemical additive is a saturated cyclic hydrocarbyl sulfate, a cyclic sultone, a tris(trihydrocarbylsilyl) phosphite, or another lithium-containing salt.

18. A solution as in claim 14 wherein the electrochemical additive is
a saturated cyclic hydrocarbyl sulfate in an amount of about 1 wt % to about 4 wt %, a cyclic sultone in an amount of about 0.5 wt % to about 4 wt %, a tris(trihydrocarbylsilyl) phosphite in an amount of about 0.15 wt % to about 1 wt %, or another lithium-containing salt in an amount of about 1 wt % to about 4 wt %, each relative to the total weight of the nonaqueous electrolyte solution; or
1,3,2-dioxathiolane 2,2-dioxide, 1-propene-1,3-sultone, 1-propane-1,3-sultone, tris(trimethylsilyl)phosphite, or lithium bis(oxolato)borate.

19. (canceled)

20. A solution as in claim 18 wherein each electrochemical additive is not used with other electrochemical additives.

21. A solution as in claim 14 wherein the electrochemical additive is selected from vinylene carbonate, 4-fluoro-ethylene carbonate, tris(trimethylsilyl)phosphite, triallyl phosphate, 1-propane-1,3-sultone, 1-propene-1,3-sultone, ethylene sulfite, 1,3,2-dioxathiolane 2,2-dioxide, 1,5,2,4-dioxadithiane 2,2,4,4-tetroxide, lithium di(fluoro)(oxolato)borate, lithium bis(oxolato)borate, and mixtures of any two or more of these.

22. A solution as in claim 21 wherein the electrochemical additive is selected from:
vinylene carbonate in an amount of about 0.5 wt % to about 3 wt %, relative to the total weight of the nonaqueous electrolyte solution;
vinylene carbonate in an amount of about 8 wt % to about 11 wt %, relative to the total weight of the nonaqueous electrolyte solution;
4-fluoro-ethylene carbonate in an amount of about 1.5 wt % to about 5 wt %, relative to the total weight of the nonaqueous electrolyte solution;
tris(trimethylsilyl)phosphite in an amount of about 0.2 wt % to about 3 wt %, relative to the total weight of the nonaqueous electrolyte solution;
triallyl phosphate in an amount of about 1 wt % to about 5 wt %, relative to the total weight of the nonaqueous electrolyte solution;
1-propane-1,3-sultone or 1-propene-1,3-sultone in an amount of about 0.5 wt % to about 4 wt %, relative to the total weight of the nonaqueous electrolyte solution;
1,3,2-dioxathiolane, 2-oxide in an amount of about 1 wt % to about 4 wt %, relative to the total weight of the nonaqueous electrolyte solution;
1,3,2-dioxathiolane 2,2-dioxide in an amount of about 1 wt % to about 4 wt %, relative to the total weight of the nonaqueous electrolyte solution;
1,5,2,4-dioxadithiane 2,2,4,4-tetroxide in an amount of about 1 wt % to about 4 wt %, relative to the total weight of the nonaqueous electrolyte solution;
lithium bis(oxolato)borate in an amount of about 1 wt % to about 4 wt %, relative to the total weight of the nonaqueous electrolyte solution; and
mixtures of any two or more of these.

23. A solution as in claim 21 wherein the electrochemical additive is
selected from 1-propane-1,3-sultone, 1-propene-1,3-sultone, 1,3,2-dioxathiolane 2,2-dioxide, tris(trimethylsilyl)phosphite, and lithium bis(oxolato)borate; or
selected from 1-propane-1,3-sultone in an amount of about 0.5 wt % to about 4 wt %, 1-propene-1,3-sultone in an amount of about 0.5 wt % to about 4 wt %, 1,3,2-dioxathiolane 2,2-dioxide, in an amount of about 1 wt % to about 4 wt %, and lithium bis(oxolato)borate in an amount of about 1 wt % to about 4 wt %, each relative to the total weight of the nonaqueous electrolyte solution.

24. (canceled)

25. A solution as in claim 23 wherein each electrochemical additive is not used with other electrochemical additives.

26. A nonaqueous lithium battery comprising a positive electrode, a negative electrode, and a nonaqueous electrolyte solution as in claim 1.

27. (canceled)

28. A solution as in claim 1 wherein the oxygen-containing brominated flame retardant of B) is
selected from the group consisting of 1-bromo-2-methoxy ethane, 1-bromo-3-methoxypropane, 2-bromo-1,1-dimethoxyethane, 2-bromo-1,4-dimethoxybenzene, 1-bromo-2-(methoxymethoxy)ethane, 1-bromovinyl ethyl ether, 1,2-dibromo-3-methoxy-1-propene, 1,2-dibromo-3-ethoxy-1-propene, and di(ethylene glycol) dibromovinyl ether; or
selected from the group consisting of 4-bromo-1,3-dioxolane, 2-bromomethyl-1,3-dioxolane, 2-dibromomethyl-1,3-dioxolane, 2-tribromomethyl-1,3-dioxolane, 2,2-bix(bromomethyl)-1,3-dioxolane, 2-(bromomethyl)-1,4-dioxane, 5,5-bis(bromomethyl)-2-methyl-1,3-dioxane, and 5,5-bis(bromomethyl)-2-ethyl-1,3-dioxane; or
selected from the group consisting of 3-bromo-2-propenyl methyl carbonate, 2,3-dibromo-2-propenyl methyl carbonate, 2,3,3-tribromo-2-propenyl methyl carbonate, 3-bromo-2-propenyl ethyl carbonate, 2,4-dibromophenyl methyl carbonate, and bis(2,3-dibromo-2-propenyl) carbonate; or
selected from the group consisting of 4-bromo-1,3-dioxol-2-one, 4,5-dibromo-1,3-dioxol-2-one, 4-bromomethyl-1,3-dioxol-2-one, 4,4-bis(bromomethyl)-1,3-dioxol-2-one, and 4,5-bis(bromomethyl)-1,3-dioxol-2-one.

29-34. (canceled)

35. A process for producing a nonaqueous electrolyte solution for a lithium battery, which process comprises combining components comprising:
i) a liquid electrolyte medium;
ii) a lithium-containing salt; and
iii) at least one oxygen-containing brominated flame retardant selected from
a) a brominated noncyclic ether,
b) a brominated cyclic diether,
c) a brominated noncyclic carbonate having two hydrocarbyl groups in which at least one hydrocarbyl group has at least one unsaturated carbon-carbon bond, or has aromatic character, and
d) a brominated cyclic carbonate having a carbonate ring, which carbonate ring has at least one unsaturated carbon-carbon bond.

36. A process as in claim 35 wherein the components further comprise at least one electrochemical additive selected from:
a) unsaturated cyclic carbonates containing three to about six carbon atoms,
b) fluorine-containing saturated cyclic carbonates containing three to about five carbon atoms and one to about four fluorine atoms,
c) tris(trihydrocarbylsilyl) phosphites containing three to about nine carbon atoms,
d) trihydrocarbyl phosphates containing three to about twelve carbon atoms,
e) cyclic sultones containing three to about eight carbon atoms,
f) saturated cyclic hydrocarbyl sulfites having a 5-membered or 6-membered ring and containing two to about six carbon atoms,
g) saturated cyclic hydrocarbyl sulfates having a 5-membered or 6-membered ring and containing two to about six carbon atoms,
h) cyclic dioxadithio polyoxide compounds having a 6-membered, 7-membered, or 8-membered ring and containing two to about six carbon atoms,
i) another lithium-containing salt, and
j) mixtures of any two or more of the foregoing.

37. (canceled)

38. A process as in claim 35 wherein the brominated flame retardant is of B), and wherein the components further comprise at least one electrochemical additive selected from vinylene carbonate, 4-fluoro-ethylene carbonate, tris(trimethylsilyl)phosphite, triallyl phosphate, 1-propane-1,3-sultone, 1-propene-1,3-sultone, ethylene sulfite, 1,3,2-dioxathiolane 2,2-dioxide, 1,5,2,4-dioxadithiane 2,2,4,4-tetroxide, lithium di(fluoro)(oxolato)borate, lithium bis(oxolato)borate, and mixtures of any two or more of these.

39. Each of the following molecules separately, as a new composition of matter:
1,2-dibromo-3-ethoxy-1-propene;
di(ethylene glycol) dibromovinyl ether;
3-bromo-2-propenyl methyl carbonate;
3-bromo-2-propenyl ethyl carbonate;
2,3-dibromo-2-propenyl methyl carbonate;
2,3,3-tribromo-2-propenyl methyl carbonate;
2,4-dibromophenyl methyl carbonate;
5,5-bis(bromomethyl)-2-methyl-1,3-dioxane;
5,5-bis(bromomethyl)-2-ethyl-1,3-dioxane;
4-bromo-1,3-dioxolane.

40-48. (canceled)
